(19) United States
(12) Patent Application Publication
(10) Pub. No.: US 2023/0310527 A1
(43) Pub. Date: Oct. 5, 2023

(54) CHINESE HERBAL MEDICINE COMPOSITION FOR IMPROVING HEARING IMPAIRMENT, AND PREPARATION METHOD AND APPLICATION THEREOF

(71) Applicant: Infinitus (China) Company Ltd., Jiangmen (CN)

(72) Inventors: Yaqian GAO, Jiangmen (CN); Yiting YANG, Jiangmen (CN); Xiaochun XIAO, Jiangmen (CN); Zhiyun DU, Jiangmen (CN); Shijing MA, Jiangmen (CN); Li LIN, Jiangmen (CN)

(73) Assignee: Infinitus (China) Company Ltd., Jiangmen (CN)

(21) Appl. No.: 17/843,398

(22) Filed: Jun. 17, 2022

(30) Foreign Application Priority Data
Mar. 30, 2022 (CN) 202210323535.7

(51) Int. Cl. A61K 36/03; A61K 36/535; A61K 36/54; A61P 27/16

(52) U.S. Cl. CPC A61K 36/03; A61K 36/535; A61K 36/54; A61P 27/16

(57) ABSTRACT

A Chinese herbal medicine composition for improving hearing impairment comprises at least two of avocado extract, ecklonia cava extract and perilla extract. The Chinese herbal medicine composition can effectively reduce a NO content secreted by cells, reduce an inflammatory reaction of cells, and have a good effect of improving LPS-induced hearing loss. Moreover, ingredients of the Chinese herbal medicine composition are medicine-and-food homologous ingredients, which have no obvious cytotoxicity and obvious side effects.

### CROSS-REFERENCE TO RELATED APPLICATION

This application claims the benefit of priority to Chinese Patent Application No. 202210323535.7, filed on Mar. 30, 2022, the entire disclosure of which is incorporated herein by reference.

### TECHNICAL FIELD

The disclosure belongs to the field of food and medicine technologies, and more particularly, relates to a Chinese herbal medicine composition for improving hearing impairment, and a preparation method and an application thereof.

### BACKGROUND

Studies have proved that noise, age and other factors may induce different degrees of hearing loss, and with the development of the disease, deafness, tinnitus and other hearing disorders may be formed, which seriously affect people's physical and mental health. In addition, oxidative stress, chronic inflammation, abnormal metabolism of neurotransmitter, abnormal metabolism of mitochondria and other factors may also promote the occurrence and development of the hearing disorders with representative symptoms such as hearing loss, deafness and tinnitus. At present, clinical treatments for hearing loss, tinnitus and deafness mainly comprise a medicine, a cochlear implant and a hearing aid, etc. Long-term administration of the medicine related to deafness such as midecamycin tablets, Erlong Zuo Ci pills, Cizhu pills and Yufeng Ningxin tablets may have certain side effects, wearing the hearing aid is palliative only, and the cochlear implant is expensive and may destroy original cochlear tissues, thus posing potential safety hazards. China is rich in medicine-and-food homologous Chinese herbal medicine resource. A large number of studies have proved that a medicine-and-food homologous Chinese herbal medicine can improve sub-health and disease states of human body, have no obvious toxic and side effects when taken, and reduce the side effects caused by the medicine. So far, there is no nutritional health food related to a hearing function on the market in China. Therefore, it is of great application significance to develop a medicine-and-food homologous food or Chinese herbal medicine preparation with a definite function of improving hearing loss and other symptoms.

Clinically, traditional Chinese medicines such as lobed kudzuvine root, fructus corni, salvia miltiorrhiza and radix rehmanniae preparata have been used for treating diseases such as deafness and tinnitus. Patent document CN1173371A discloses a Chinese medicine for treating deafness, which comprises 19 Chinese medicinal materials such as lobed kudzuvine root, salvia miltiorrhiza, radix bupleuri, rhizoma acori tatarinowii , radix rehmanniae preparata, flos lonicerae japonicae and flos chrysanthemi indici. The Chinese medicine has functions of opening and closing orifices, activating blood circulation, dredging collaterals, nourishing kidneys and improving hearing, and is mainly used for treating sudden deafness, nervous deafness, presbycusis and toxic deafness. Clinical verification by 848 cases has shown that a cure rate ranges from 38.2% to 67.2%, so that an effective treatment rate is not high. Patent document CN106620011A discloses a Chinese medicinal composition for treating deafmutism or deafness. An ear drop is prepared with flos lonicerae, fructus trichosanthis, Chinese angelica, rhizoma corydalis, ligusticum wallichii, ginseng, trogopterus dung, synthetic borneol, moschus or artificial moschus, magnetite, fructus lycii, taraxacum and common macrocarpium fruit, which can promote the regeneration and repair of damaged and dead sensory mother cells and conductive nerves, thus improving hearing. Studies have proved that avocado is rich in protein, fat, carbohydrate, ash, vitamins, and mineral elements such as sodium, potassium, magnesium and calcium, thus having functions of anti-oxidation, blood lipid reduction, blood pressure reduction and nerve protection. As a brown algae, ecklonia cava is rich in polyphenols, and has biological activities such as anti-oxidation, anti-inflammation and anti-tumor. Perilla leaf is a traditional Chinese medicine for clearing away heat and toxic materials, has effects of relieving exterior syndrome and dispelling cold, ventilating lung and resolving phlegm, and promoting qi circulation and regulating stomach, and is widely used for treating inflammatory diseases.

### SUMMARY

An object of the disclosure in a first aspect is providing a Chinese herbal medicine composition.

An object of the disclosure in a second aspect is providing a preparation method of the Chinese herbal medicine composition in the first aspect.

An object of the disclosure in a third aspect is providing an application of the Chinese herbal medicine composition in the first aspect in preparing a product.

An object of the disclosure in a fourth aspect is providing a product.

An object of the disclosure in a fifth aspect is providing an application of the Chinese herbal medicine composition in the first aspect and/or the product in the fourth aspect in treating hearing impairment.

In order to achieve the objects above, the technical solutions used in the disclosure are as follows.

In a first aspect of the disclosure, a Chinese herbal medicine composition is provided, and the Chinese herbal medicine composition is any one of (b1) to (b4):
(b1) comprising the following ingredients in parts by weight: 50 parts to 100 parts of avocado extract and 20 parts to 100 parts of ecklonia cava extract;
(b2) comprising the following ingredients in parts by weight: 50 parts to 100 parts of avocado extract and 20 parts to 100 parts of perilla extract;
(b3) comprising the following ingredients in parts by weight: 20 parts to 100 parts of ecklonia cava extract and 20 parts to 100 parts of perilla extract; and
(b4) comprising the following ingredients in parts by weight: 50 parts to 100 parts of avocado extract, 20 parts to 100 parts of perilla extract and 20 parts to 100 parts of ecklonia cava extract.

Preferably, the Chinese herbal medicine composition is any one of (c1) to (c4):
(c1) comprising the following ingredients in parts by weight: 60 parts to 90 parts of avocado extract and 30 parts to 90 parts of ecklonia cava extract;
(c2) comprising the following ingredients in parts by weight: 60 parts to 90 parts of avocado extract and 30 parts to 90 parts of perilla extract;
(c3) comprising the following ingredients in parts by weight: 30 parts to 90 parts of ecklonia cava extract and 30 parts to 90 parts of perilla extract; and
(c4) comprising the following ingredients in parts by weight: 60 parts to 90 parts of avocado extract, 30 parts to 90 parts of perilla extract and 30 parts to 90 parts of ecklonia cava extract.

Preferably, the Chinese herbal medicine composition is any one of (d1) to (d4):
(d1) comprising the following ingredients in parts by weight: 70 parts to 90 parts of avocado extract and 60 parts to 90 parts of ecklonia cava extract;
(d2) comprising the following ingredients in parts by weight: 70 parts to 90 parts of avocado extract and 70 parts to 90 parts of perilla extract;
(d3) comprising the following ingredients in parts by weight: 60 parts to 90 parts of ecklonia cava extract and 70 parts to 90 parts of perilla extract; and
(d4) comprising the following ingredients in parts by weight: 70 parts to 90 parts of avocado extract, 70 parts to 90 parts of perilla extract and 60 parts to 90 parts of ecklonia cava extract.

Preferably, the avocado extract is an avocado water extract.

Preferably, the ecklonia cava extract is an ecklonia cava alcohol extract.

Preferably, the perilla extract is a perilla alcohol extract.

Preferably, the perilla extract is a perilla leaf extract.

Preferably, the content of an unsaturated fatty acid in the avocado extract ranges from 60 wt % to 80 wt %; and further ranges from 69.8 wt % to 78.6 wt %.

Preferably, the content of a polyphenol in the ecklonia cava extract ranges from 5 w t% to 25 wt %; and further ranges from 15 wt % to 20 wt %.

In a second aspect of the disclosure, a preparation method of the Chinese herbal medicine composition in the first aspect of the disclosure is provided, and the preparation method comprises: mixing the ingredients in the Chinese herbal medicine composition in the first aspect of the disclosure to obtain the Chinese herbal medicine composition.

In a third aspect of the disclosure, an application of the Chinese herbal medicine composition in the first aspect of the disclosure in preparing a product is provided.

Preferably, the product comprises at least one of food and medicine.

Preferably, the food is a functional food.

Preferably, the food comprises at least one of beverage, biscuit, candy and cake.

Preferably, the beverage is a solid beverage.

Preferably, a dosage form of the medicine comprises at least one of capsule, tablet, granule, mixture, decoction, pill, electuary, dripping pill, medicinal tea, tincture, and medicinal liquor.

Preferably, the product further comprises a pharmaceutically acceptable adjuvant or a food-acceptable adjuvant.

Preferably, the product has at least one function of (e1) to (e4):
(e1) preventing and/or treating hearing impairment;
(e2) reducing an ABR value;
(e3) inhibiting generation of an inflammatory factor; and
(e4) reducing a MDA content.

Preferably, the inflammatory factor comprises at least one of TNF-α and NO; and further preferably, the inflammatory factor comprises NO.

Preferably, the hearing impairment comprises at least one of labyrinthitis, hearing loss, hearing disorder, tinnitus and deafness.

Preferably, the hearing loss comprises at least one of aging hearing loss, noise hearing loss and inflammatory hearing loss; and further preferably, the hearing loss comprises the noise hearing loss.

In a fourth aspect of the disclosure, a product comprising the Chinese herbal medicine composition in the first aspect of the disclosure is provided.

Preferably, the product comprises at least one of food and medicine.

Preferably, the food is a functional food.

Preferably, the food comprises at least one of beverage, biscuit, candy and cake.

Preferably, the beverage is a solid beverage.

Preferably, a dosage form of the medicine comprises at least one of capsule, tablet, granule, mixture, decoction, pill, electuary, dripping pill, medicinal tea, tincture, and medicinal liquor.

Preferably, the product further comprises a pharmaceutically acceptable adjuvant or a food-acceptable adjuvant.

Preferably, the product has at least one function of (e1) to (e4):
(e1) preventing and/or treating hearing impairment;
(e2) reducing an ABR value;
(e3) inhibiting generation of an inflammatory factor; and
(e4) reducing a MDA content.

Preferably, the inflammatory factor comprises at least one of TNF-α and NO; and further preferably, the inflammatory factor comprises NO.

Preferably, the hearing impairment comprises at least one of labyrinthitis, hearing loss, hearing disorder, tinnitus and deafness.

Preferably, the hearing loss comprises at least one of aging hearing loss, noise hearing loss and inflammatory hearing loss; and further preferably, the hearing loss comprises the noise hearing loss.

In a fifth aspect of the disclosure, an application of the Chinese herbal medicine composition in the first aspect and/or the product in the fourth aspect of the disclosure in treating hearing impairment is provided.

Preferably, the application comprises the following step of: administering an effective amount of the Chinese herbal medicine composition in the first aspect and/or the product in the fourth aspect to a patient.

Preferably, an administration dosage of the Chinese herbal medicine composition ranges from 0.5 g/d to 4.0 g/d.

Preferably, an administration dosage of the product ranges from 0.5 g/d to 4.0 g/d based on the Chinese herbal medicine composition.

Preferably, a method of administrating the Chinese herbal medicine composition comprises at least one of oral administration, sublingual administration, intravenous injection, intramuscular injection, subcutaneous injection, inhalation and transdermal administration.

Preferably, a method of administrating the product comprises at least one of oral administration, sublingual administration, intravenous injection, intramuscular injection, subcutaneous injection, inhalation and transdermal administration.

Preferably, the hearing impairment comprises at least one of labyrinthitis, hearing loss, hearing disorder, tinnitus and deafness.

Preferably, the hearing loss comprises at least one of aging hearing loss, noise hearing loss and inflammatory hearing loss; and further preferably, the hearing loss comprises the noise hearing loss.

The disclosure has the beneficial effects as follows:

The disclosure discloses the Chinese herbal medicine composition for improving hearing impairment. The Chinese herbal medicine composition can effectively reduce a NO content secreted by cells, reduce an inflammatory reaction of cells, and have a good effect of improving LPS-induced hearing loss. In addition, the Chinese herbal medicine composition is not a simple combination, but has a synergistic effect in anti-inflammation. Meanwhile, the Chinese herbal medicine composition can reduce an ABR value and a MDA content of a patient suffering from hearing loss, and have a synergistic effect in reducing the ABR value and the MDA content, thus significantly improving hearing impairment, and can be used as an active ingredient to be developed into functional food and medicine for preventing and/or treating the hearing impairment (labyrinthitis, hearing loss, hearing disorder, tinnitus and deafness. Moreover, ingredients of the Chinese herbal medicine composition are medicine-and-food homologous ingredients, which have no obvious cytotoxicity and obvious side effects. The Chinese herbal medicine composition can be taken with medicines for a long time, and can reduce chronic side effects possibly caused by clinical medicines while cooperating with the clinical medicines to exert effects of the medicines, thus being beneficial for recovering health of body, and improving a treatment effect.

### DETAILED DESCRIPTION

The contents of the disclosure are further described in detail hereinafter with reference to the specific embodiments.

It should be understood that these embodiments are only used for describing the disclosure and are not intended to limit the scope of the disclosure.

If the detailed conditions for the experimental method are not indicated in the following embodiments, the conventional conditions or the conditions suggested by the manufacturers should be followed. Unless otherwise specified, the materials and reagents used in the embodiments are all commercially available reagents and materials.

An avocado extract in the embodiment is a low-temperature pressed extract (water extract), which is purchased from Guangzhou Kanglun Biotechnology Co., Ltd., with an article number (batch number) of 202103001. Unsaturated fatty acid contents in the embodiments are different due to different batches. A preparation method of the avocado extract comprises: peeling and coring an avocado, collecting fresh, weighing a proper amount of fresh, pulping the fresh according to a ratio of fresh to water of 1:2, processing a pulp with a colloid mill for 1 minute, adding water according to 1:2 to clean the machine, mixing the pulp with the cleaning liquid, stirring the mixture uniformly, then adjusting a pH value to be 8.0, then stirring the mixture at a low speed for 1.5 hours in water bath at 75° C., then centrifuging the mixture at 10,000 r/min for 10 minutes, then refrigerating upper-layer oil (comprising an emulsified layer) in a refrigerator at 4° C. for 24 hours, then centrifuging the upper-layer oil in a low-temperature and high-speed centrifuge at 10,000 r/min for 10 minutes to remove the emulsified layer and take upper-layer clear oil, and collecting and storing the avocado oil in the refrigerator at 4° C. to obtain the avocado extract.

An ecklonia cava extract is 50 wt % ethanol extract, which is purchased from Guangzhou Kanglun Biotechnology Co., Ltd., with an article number (batch number) of 202104002. Polyphenol contents in the embodiments are different due to different batches. A preparation method of the ecklonia cava extract comprises: taking a raw material of a dried ecklonia cava product, adding 12 times and 10 times of 50% ethanol in sequence, refluxing the mixture at 65° C. for 60 minutes and 50 minutes respectively, filtering the mixture with 300 meshes, recovering ethanol, decompressing and concentrating the solution at 60° C. into 1 g/mL concentrated solution containing an original medicinal material, and subjecting the concentrated solution to spray drying to obtain the ecklonia cava extract.

A perilla leaf extract is 50 wt % ethanol extract, which is purchased from Guangzhou Kanglun Biotechnology Co., Ltd., with an article number of 202101006. A preparation method of the perilla leaf extract comprises: taking a raw material of a dried perilla leaf product, adding 12 times and 10 times of 50% ethanol in sequence, refluxing the mixture at 65° C. for 40 minutes and 35 minutes respectively, filtering the mixture with 300 meshes, recovering ethanol, decompressing and concentrating the solution at 60° C. into 1 g/mL concentrated solution containing an original medicinal material, and subjecting the concentrated solution to spray drying to obtain the perilla leaf extract.

Preferably, the preparation method of the avocado extract comprises: mixing an avocado with water, pulping the mixture, adjusting a pH value to be 7.5 to 9, stirring the mixture at 65° C. to 85° C. for 1 hour to 2 hours, centrifuging the mixture, refrigerating upper-layer oil at 2° C. to 6° C. for 20 hours to 28 hours, centrifuging the upper-layer oil, and taking upper-layer clear oil to obtain the avocado extract.

Preferably, the avocado is avocado fresh.

Preferably, the centrifuging is carried out at 8,000 r/min to 12,000 r/min for 8 minutes to 12 minutes.

Preferably, the preparation method of the ecklonia cava extract comprises: extracting ecklonia cava with 40% to 60% ethanol to obtain the ecklonia cava extract. The specific method comprises: extracting the ecklonia cava with 11 times to 13 times and 9 times to 11 times of 40% to 60% ethanol in sequence, subjecting the mixture to solid-liquid separation, removing ethanol, and concentrating and drying the solution to obtain the ecklonia cava extract.

Preferably, the extracting is reflux extraction.

Preferably, the reflux extraction is carried out at 60° C. to 70° C. for 45 minutes to 65 minutes.

Preferably, the concentrating is to concentrate to 0.5 g/ml to 1.5 g/ml based on the ecklonia cava.

Preferably, the ecklonia cava is a dried ecklonia cava product.

Preferably, the preparation method of the perilla extract comprises: extracting a perilla with 40% to 60% ethanol to obtain the perilla extract. The specific method comprises: extracting the perilla with 11 times to 13 times and 9 times to 11 times of 40% to 60% ethanol in sequence, subjecting the mixture to solid-liquid separation, removing ethanol, and concentrating and drying the solution to obtain the perilla extract.

Preferably, the perilla is perilla leaf.

Preferably, the extracting is reflux extraction.

Preferably, the reflux extraction is carried out at 60° C. to 70° C. for 45 minutes to 65 minutes.

Preferably, the concentrating is to concentrate to 0.5 g/ml to 1.5 g/ml based on the perilla leaf.

Preferably, the perilla is a dried perilla product.

### EXAMPLE 1: A CHINESE HERBAL MEDICINE COMPOSITION FOR IMPROVING HEARING IMPAIRMENT

1. A Chinese herbal medicine composition for improving hearing impairment was composed of the following ingredients in parts by weight: 90 parts of avocado extract, 90 parts of ecklonia cava extract, and 90 parts of perilla leaf extract, wherein the content of an unsaturated fatty acid in the avocado extract was 78.6 wt %, and the content of a polyphenol in the ecklonia cava extract was 15 wt %.

2. A preparation method of the Chinese herbal medicine composition for improving hearing impairment above was mixing the avocado extract, the ecklonia cava extract and the perilla leaf extract to obtain the Chinese herbal medicine composition.

### EXAMPLE 2: A CHINESE HERBAL MEDICINE COMPOSITION FOR IMPROVING HEARING IMPAIRMENT

1. A Chinese herbal medicine composition for improving hearing impairment was composed of the following ingredients in parts by weight: 60 parts of avocado extract, 30 parts of ecklonia cava extract, and 30 parts of perilla leaf extract, wherein the content of an unsaturated fatty acid in the avocado extract was 78.6 wt %, and the content of a polyphenol in the ecklonia cava extract was 15 wt %.

2. A preparation method of the Chinese herbal medicine composition for improving hearing impairment above was mixing the avocado extract, the ecklonia cava extract and the perilla leaf extract to obtain the Chinese herbal medicine composition.

### EXAMPLE 3: A CHINESE HERBAL MEDICINE COMPOSITION FOR IMPROVING HEARING IMPAIRMENT

1. A Chinese herbal medicine composition for improving hearing impairment was composed of the following ingredients in parts by weight: 70 parts of avocado extract, 60 parts of ecklonia cava extract, and 70 parts of perilla leaf extract, wherein the content of an unsaturated fatty acid in the avocado extract was 69.8 wt %, and the content of a polyphenol in the ecklonia cava extract was 20 wt %.

2. A preparation method of the Chinese herbal medicine composition for improving hearing impairment above was mixing the avocado extract, the ecklonia cava extract and the perilla leaf extract to obtain the Chinese herbal medicine composition.

### EXAMPLE 4: A CHINESE HERBAL MEDICINE COMPOSITION FOR IMPROVING HEARING IMPAIRMENT

1. A Chinese herbal medicine composition for improving hearing impairment was composed of the following ingredients in parts by weight: 60 parts of avocado extract, 70 parts of ecklonia cava extract, and 65 parts of perilla leaf extract, wherein the content of an unsaturated fatty acid in the avocado extract was 69.8 wt %, and the content of a polyphenol in the ecklonia cava extract was 20 wt %.

2. A preparation method of the Chinese herbal medicine composition for improving hearing impairment above was mixing the avocado extract, the ecklonia cava extract and the perilla leaf extract to obtain the Chinese herbal medicine composition.

### EXAMPLE 5: A CHINESE HERBAL MEDICINE COMPOSITION FOR IMPROVING HEARING IMPAIRMENT

1. A Chinese herbal medicine composition for improving hearing impairment was composed of the following ingredients in parts by weight: 90 parts of avocado extract and 90 parts of ecklonia cava extract, wherein the content of an unsaturated fatty acid in the avocado extract was 78.6 wt %, and the content of a polyphenol in the ecklonia cava extract was 15 wt %.

2. A preparation method of the Chinese herbal medicine composition for improving hearing impairment above was mixing the avocado extract and the ecklonia cava extract to obtain the Chinese herbal medicine composition.

### EXAMPLE 6: A CHINESE HERBAL MEDICINE COMPOSITION FOR IMPROVING HEARING IMPAIRMENT

1. A Chinese herbal medicine composition for improving hearing impairment was composed of the following ingredients in parts by weight: 90 parts of ecklonia cava extract and 90 parts of perilla leaf extract, wherein the content of a polyphenol in the ecklonia cava extract was 15 wt %.

2. A preparation method of the Chinese herbal medicine composition for improving hearing impairment above was mixing the ecklonia cava extract and the perilla leaf extract to obtain the Chinese herbal medicine composition.

### EXAMPLE 7: A CHINESE HERBAL MEDICINE COMPOSITION FOR IMPROVING HEARING IMPAIRMENT

1. A Chinese herbal medicine composition for improving hearing impairment was composed of the following ingredients in parts by weight: 90 parts of avocado extract and 90 parts of perilla leaf extract, wherein the content of an unsaturated fatty acid in the avocado extract was 78.6 wt %.

2. A preparation method of the Chinese herbal medicine composition for improving hearing impairment above was mixing the avocado extract and the perilla leaf extract to obtain the Chinese herbal medicine composition.

### Comparative Example 1: a Chinese Herbal Medicine Extract for improving hearing Impairment

1. A Chinese herbal medicine extract for improving hearing impairment was composed of the following ingredient in parts by weight: 90 parts of avocado extract, wherein the content of an unsaturated fatty acid in the avocado extract was 78.6 wt %.

### Comparative Example 2: a Chinese Herbal Medicine Extract for Improving Hearing Impairment

1. A Chinese herbal medicine extract for improving hearing impairment was composed of the following ingredient in parts by weight: 90 parts of perilla leaf extract.

### Comparative Example 3: a Chinese Herbal Medicine Extract for Improving Hearing Impairment

1. A Chinese herbal medicine extract for improving hearing impairment was composed of the following ingredient in parts by weight: 90 parts of ecklonia cava extract, wherein the content of a polyphenol in the ecklonia cava extract was 15 wt %.

### Effect Example

### 1. Influence of Chinese Herbal Medicine Extract/Chinese Herbal Medicine Composition on Nitric Oxide Secretion of Raw264.7 Cells Induced by Lipopolysaccharide (LPS)

After a lipopolysaccharide (LPS) inducer acted on a cochlea, cochlear tissues were stimulated to cause a series of inflammatory reactions. The lipopolysaccharide inducer promoted over-expression of inflammatory factors such as TNF-α and NO while inducing hearing loss of the cochlea, and induced an oxidative stress reaction of the cochlear tissues, which aggravated the hearing loss. Therefore, under a modeling condition, abnormal nitric oxide (NO) secretion could also reflect a degree of hearing loss in terms of inflammation.

According to the method in the reference document [Liu Guanting, Hu Liuyun, Ma Shijing, Han Ping, Lin Li, Du Zhiyun, et al. Process optimization and anti-inflammatory activity of the curcumin emulsion [J]. China Surfactant Detergent & Cosmetics, 2021, 51(08): 741-747], a mouse macrophage cell line RAW264.7 was cultured in a DMEM complete medium comprising 10 v/v % FBS fetal bovine serum and 1 v/v % double antibiotics (100 mg·mL⁻¹ streptomycin and 100 U·mL⁻¹ penicillin). After a culture flask was full of cells, the cells were subcultured according to a dilution ratio of 1:3 by volume. RAW264.7 cells in logarithmic growth phase were added into a 96-well plate by 8×10³ cells/well, and incubated overnight at 37° C. under 5% CO₂. In a sample group, samples (final concentrations of the Chinese herbal medicine composition in Example 1 and the Chinese herbal medicine extracts in Comparative examples 1 to 3 were 10 μg·mL⁻¹, 50 μg·mL⁻¹ and 100 μg·mL⁻¹) were added to process for 24 hours (in a blank control group, no sample was added, and each processing was repeated for 3 times), 20 μL of 5 mg·mL¹ MTT solution was added into each well, and incubated in an incubator in the dark for 4 hours, then an upper-layer medium was discarded, 100 μL of DMSO solution was added, and oscillated in the dark for 10 minutes, an absorbance value was measured at 492 nm with a microplate reader, and a cell activity was calculated, wherein the cell activity (%)=A_{sample group}/A_{blank control group}. Results are shown in Table 1: survival rates of RAW264.7 under the avocado extract in Comparative example 1 at different concentrations (10 μg·mL⁻¹, 50 μg·mL⁻¹, and 100 μg·mL⁻¹), the perilla leaf extract in Comparative example 2 at different concentrations (10 μg·mL⁻¹, 50 μg·mL⁻¹, and 100 μg·mL⁻¹), the ecklonia cava extract in Comparative example 3 at different concentrations (10 μg·mL⁻¹, 50 μg·mL⁻¹, and 100 μg·mL⁻¹), and the Chinese herbal medicine composition in Example 1 at different concentrations (10 μg·mL⁻¹, 50 μg·mL⁻¹, and 100 μg·mL⁻¹) are all greater than 90%. It can be seen that the Chinese herbal medicine extract/Chinese herbal medicine composition at the concentrations above does not influence normal growth of the RAW264.7 cells, and has no obvious cytotoxicity.

TABLE 1
Influences of samples on activity of RAW264.7 cells

| | Concentration/ | Absorbance/ | Cell |
|---|---|---|---|
| Raw material | (μg/mL) | (A) | activity/(%) |
| Blank control group | — | 1.041 | 100 |
| Comparative example 1 | 100 | 0.994 ± 0.025 | 95.56 ± 3.73 |
| (avocado extract) | 50 | 0.967 ± 0.016 | 92.90 ± 1.41 |
| | 10 | 0.949 ± 0.022 | 91.17 ± 2.59 |
| Comparative example 2 | 100 | 0.939 ± 0.024 | 90.25 ± 1.11 |
| (perilla leaf extract) | 50 | 0.948 ± 0.020 | 91.11 ± 1.95 |
| | 10 | 0.967 ± 0.037 | 92.97 ± 3.60 |
| Comparative example 3 | 100 | 0.961 ± 0.039 | 92.36 ± 2.43 |
| (ecklonia cava extract) | 50 | 0.979 ± 0.015 | 94.12 ± 1.55 |
| | 10 | 1.021 ± 0.027 | 98.13 ± 2.11 |
| Example 1 (Chinese | 100 | 0.960 ± 0.021 | 92.23 ± 0.31 |
| herbal medicine | 50 | 1.017 ± 0.024 | 97.71 ± 4.79 |
| composition) | 10 | 0.996 ± 0.012 | 95.76 ± 2.32 |

Cell experimental groups were divided into 12 groups, respectively comprising a blank group, a model group, and sample groups (a group of avocado extract (Comparative example 1), a group of ecklonia cava extract (Comparative example 3), a group of perilla leaf extract (Comparative example 2) and groups of Examples 1 to 7). RAW264.7 cell lines in logarithmic growth phase were inoculated on a 6-well plate at a density of 2×10⁶ cells/mL, and then incubated in an incubator for 24 hours before administration: a DMEM complete medium was added in the blank group, LPS with a final mass concentration of 1 mg·mL⁻¹ was added in the model group, and samples with final mass concentrations of 10 μg·mL⁻¹, 50 μg·mL⁻¹ and 100 μg·mL⁻¹ (the Chinese herbal medicine compositions in Examples 1 to 7 and the Chinese herbal medicine extracts in Comparative examples 1 to 3) and 1 mg·mL-⁻¹ LPS were respectively added in the sample groups, so that each group had a total volume of 3 mL, and was continuously cultured in a cell incubator for 24 hours (each processing was repeated for 3 times). A cell supernatant was collected, and measured strictly according to an instruction of a NO detection kit (purchased from Beyotime, S0021S): a standard solution NaNO₂ with an original concentration of 1 mol·L⁻¹ was diluted with a complete medium as a diluent. 50 μL of control sample solution and sample at each concentration, 50 μL of Griess Reagent I solution and 50 μL of Griess Reagent II solution were added into a 96-well plate according to 150 μL/well, and incubated at 37° C. for 10 minutes. An absorbance of each well was measured at 540 nm with a microplate reader and recorded after oscillation on an oscillator for 5 minutes, a NO content was calculated, and a NO inhibition ratio (IR) was calculated according to the following formula, wherein S was the NO content of each group.

$$IR ( \% ) = \frac{( {S}_{model group} - {S}_{s ample group} )}{( {S}_{model lgroup} - {S}_{blank group} )} \times 1 \; 00 \% .$$

The synergistic index of each ingredient was calculated (NO was calculated) by an index-value content (which was namely an average value of the inhibition rate). A calculation formula in a calculation reference document (Berenbaum M C. The expected effect of a combination of agents: the general solution. Journal of Theoretical Biology, 1985, 114: 413-431.) of a synergistic interaction index (Berenbaum index) was:

$${\sum }_{i = 1}^{n} \frac{Xi}{Xie} ,$$

wherein Xi was a dosage of an ith medicine in medicine combination; Xie was the dosage of the ith medicine in separate application capable of achieving the same effect as that in the medicine combination; and n was a number of medicines in the medicine combination. When the Berenbaum index was less than 1, the medicine combination showed a synergistic effect, while when the Berenbaum index was greater than 1, the medicine combination showed an antagonistic effect.

Experimental results are shown in Tables 2 and 3: the LPS induces abnormal nitric oxide secretion after acting on the RAW264.7 cells, and the NO content of cells in the model group is increased significantly, which indicates successful modeling. After the Chinese herbal medicine extracts in Comparative examples 1 to 3 and the Chinese herbal medicine compositions in Examples 1 to 7 act on the LPS-modeled cells, the secreted NO content of the cells can be effectively reduced, and the inflammatory reaction of the cells can be reduced, which indicates that the Chinese herbal medicine extracts in Comparative examples 1 to 3 and the Chinese herbal medicine compositions in Examples 1 to 7 have a good effect on improving the LPS-induced hearing loss, with a mechanism that a degree of hearing loss can be reduced by inhibiting an inflammation process. Moreover, the Chinese herbal medicine compositions in Examples 1 to 7 have a better inhibiting effect on the LPS-induced NO secretion of the RAW264.7 cells than the Chinese herbal medicine extracts in Comparative examples 1 to 3. In addition, the synergistic indexes of the Chinese herbal medicine compositions (10 μg/mL and 50 μg/mL) in Examples 1 to 7 are all less than 1, which indicates that the Chinese herbal medicine compositions in Examples 1 to 7 have the synergistic effect, but are not a simple combination, thus further reflecting synergistic anti-inflammatory interaction benefits of the combination.

TABLE 2
Inhibiting effect of Chinese herbal medicine compositions/Chinese herbal
medicine extracts on LPS-induced NO secretion in RAW264.7 cells

| | Concentration/ | Absorbance/ | NO content/ | Inhibition rate/ |
|---|---|---|---|---|
| Experimental group | (μg/mL) | (A) | (μmol/L) | (%) |
| Blank control group | — | 0.095 ± 0.002 | 0.34 ± 0.06 | — |
| Model group | LPS-1 mg/mL | 0.168 ± 0.004 | 12.31 ± 0.16 | — |
| Example 1 | 100 | 0.113 ± 0.006 | 3.35 ± 0.29 | 74.89 ± 4.94 |
| | 50 | 0.124 ± 0.003 | 5.04 ± 0.25 | 60.73 ± 2.09 |
| | 10 | 0.136 ± 0.001 | 7.12 ± 0.11 | 43.38 ± 2.19 |
| Example 2 | 100 | 0.122 ± 0.003 | 4.83 ± 0.14 | 62.56 ± 3.45 |
| | 50 | 0.138 ± 0.002 | 7.34 ± 0.51 | 41.55 ± 5.54 |
| | 10 | 0.149 ± 0.002 | 9.25 ± 0.19 | 25.57 ± 2.87 |
| Example 3 | 100 | 0.111 ± 0.003 | 2.97 ± 0.09 | 78.08 ± 1.37 |
| | 50 | 0.125 ± 0.005 | 5.21 ± 0.16 | 59.36 ± 3.45 |
| | 10 | 0.145 ± 0.001 | 8.60 ± 0.05 | 31.05 ± 3.07 |
| Example 4 | 100 | 0.119 ± 0.002 | 4.33 ± 0.03 | 66.67 ± 1.58 |
| | 50 | 0.129 ± 0.003 | 5.92 ± 0.14 | 53.42 ± 0.79 |
| | 10 | 0.146 ± 0.004 | 8.65 ± 0.49 | 30.59 ± 2.09 |
| Example 5 | 100 | 0.150 ± 0.002 | 9.42 ± 0.25 | 24.20 ± 3.45 |
| | 50 | 0.151 ± 0.002 | 9.47 ± 0.38 | 23.74 ± 1.58 |
| | 10 | 0.154 ± 0.004 | 9.96 ± 0.23 | 19.63 ± 5.54 |
| Example 6 | 100 | 0.147 ± 0.005 | 8.81 ± 0.11 | 29.22 ± 0.79 |
| | 50 | 0.146 ± 0.001 | 8.76 ± 0.29 | 29.68 ± 2.19 |
| | 10 | 0.151 ± 0.003 | 9.91 ± 0.32 | 20.08 ± 3.45 |
| Example 7 | 100 | 0.132 ± 0.002 | 6.46 ± 0.02 | 48.86 ± 1.58 |
| | 50 | 0.143 ± 0.005 | 8.16 ± 0.53 | 34.70 ± 1.58 |
| | 10 | 0.153 ± 0.006 | 9.85 ± 0.28 | 20.55 ± 5.48 |
| Comparative | 100 | 0.147 ± 0.002 | 8.92 ± 0.18 | 28.31 ± 4.11 |
| example 1 | 50 | 0.151 ± 0.005 | 9.58 ± 0.12 | 22.83 ± 4.40 |
| | 10 | 0.157 ± 0.002 | 10.45 ± 0.33 | 15.53 ± 2.09 |
| Comparative | 100 | 0.138 ± 0.003 | 7.39 ± 0.38 | 41.10 ± 3.45 |
| example 2 | 50 | 0.148 ± 0.004 | 8.98 ± 0.28 | 27.85 ± 3.16 |
| | 10 | 0.153 ± 0.006 | 9.91 ± 0.37 | 20.09 ± 4.40 |
| Comparative | 100 | 0.155 ± 0.002 | 10.18 ± 0.22 | 17.81 ± 1.37 |
| example 3 | 50 | 0.158 ± 0.002 | 10.73 ± 0.49 | 13.24 ± 2.28 |
| | 10 | 0.161 ± 0.001 | 11.11 ± 0.53 | 10.05 ± 0.57 |

TABLE 3
Synergistic indexes of examples

| | Synergistic index | | |
|---|---|---|---|
| Experimental group | 100 μg/mL | 50 μg/mL | 10 μg/mL |
| Comparative example 1 | | | |
| Comparative example 2 | | | |
| Comparative example 3 | | | |
| Example 1 | 0.3878 | 0.1755 | 0.0351 |
| Example 2 | 0.4643 | 0.2564 | 0.0595 |
| Example 3 | 0.3720 | 0.1795 | 0.0490 |
| Example 4 | 0.4356 | 0.1995 | 0.0497 |
| Example 5 | 0.9529 | 0.3798 | 0.0652 |
| Example 6 | 1.0080 | 0.3461 | 0.3298 |
| Example 7 | 0.7103 | 0.3651 | 0.0867 |

### 2. Noise-Induced Hearing Loss

A noise-modeled animal model was used for evaluation, After 7 days of adaptive feeding, SD rats were divided into a blank group, a model group, a positive group (Zuo Ci Erlong pills) and sample groups (Examples 1 to 7 and Comparative examples 1 to 3), and there were 13 groups in total, with 6 animals in each group. Except the blank group, the SD rats were put in cages in the remaining model group, positive group and sample groups respectively, with 6 SD rats in each cage, and placed in an exposure cabin (a soundproof box with a length of 1.2 m, a width of 1.2 m and a height of 1.7 m), and a noise was generated by a white noise generator, amplified by an A-K200 power amplifier, and played to the exposure cabin by a speaker located on a top portion of the exposure cabin. When exposed, the noise was continuously monitored by a sound pressure meter, and the noise was kept at a frequency of 20 Hz to 20 kHz. An average sound intensity was a sound pressure level of 90 dB, and a sound propagation unevenness in an exposure range of the animals was (±2) dB. In the case of noise exposure, ventilation in the exposure cabin was ensured, and the SD rats were fed normally and drunk water normally, and were continuously exposed for 6 hours every day, lasting for 14 days in total. During modeling, the rats were subjected to intragastric administration according to a dosage of 100 mg/kg (according to an intragastric administration dosage of 0.3 mL/100 g by rat body weight per day) in the positive group and the sample groups, and the rats were subjected to intragastric administration with the same amount of normal saline in the blank group and the model group. At the end of the experiment (on the 14^{th} day), ABR values of the rats in the groups at 8 kHz were tested by an auditory brainstem responsor (ABR) instrument. After finishing testing, cochlear tissues of the rats in the groups were taken, and malondialdehyde (MDA) index contents were tested by an ELISA method. Results are shown in Table 4: compared with the blank group, the ABR value and the MDA value of the rats in the model group are both significantly different (p<0.001), which indicates successful modeling. Compared with the model group, the ABR values of the groups of Examples 1 to 5 and the group of Example 7 are reduced significantly, and the MDA values of the groups of Examples 1 to 7 are reduced significantly, while compared with the model group, the ABR values and the MDA values of the groups of Comparative examples 1 to 3 have no significant difference, which indicates that the Chinese herbal medicine compositions in Examples 1 to 7 are better than the Chinese herbal medicine extracts in Comparative examples 1 to 3 in reducing the ABR value and the MDA value. It can be seen that the Chinese herbal medicine composition provided by the disclosure is not a simple combination, and the Chinese herbal medicine composition has an improved effect of reducing the ABR value and the MDA value. Meanwhile, the MDA values of the Chinese herbal medicine compositions in Examples 1 to 7 are lower than that of the positive group, and the ABR values of the Chinese herbal medicine compositions in Examples 1 to 4 are lower than that of the positive group, which indicates that the Chinese herbal medicine composition provided by the present application has a better effect of treating the noise-induced hearing loss than the Zuo Ci Erlong pills.

TABLE 4
Experimental results of noise-induced hearing loss of rats

| Experimental group | ABR value/(dB) | MDA/(U/mL) |
|---|---|---|
| Blank group | 30.00 ± 5.00 | 16.80 ± 1.10 |
| Model group | 44.71 ± 2.05^{###} | 21.46 ± 1.48^{###} |
| Positive group | 34.98 ± 1.23*** | 20.40 ± 0.80 |
| Group of Example 1 | 30.00 ± 3.16*** | 16.22 ± 0.95*** |
| Group of Example 2 | 34.00 ± 4.18*** | 7.03 ± 0.99** |
| Group of Example 3 | 33.33 ± 2.58*** | 15.16 ± 3.12*** |
| Group of Example 4 | 31.00 ± 12.81*** | 15.32 ± 3.55*** |
| Group of Example 5 | 37.50 ± 2.89* | 18.48 ± 0.81* |
| Group of Example 6 | 39.27 ± 1.17 | 17.31 ± 1.29* |
| Group of Example 7 | 37.50 ± 2.74* | 18.21 ± 0.99* |
| Group of Comparative | 40.83 ± 4.92 | 18.46 ± 1.26 |
| example 1 | | |
| Group of Comparative | 38.50 ± 4.26 | 18.89 ± 1.10 |
| example 2 | | |
| Group of Comparative | 42.50 ± 2.74 | 19.38 ± 1.52 |
| example 3 | | |

Note:
^{###}indicates “compared with the blank group, p < 0.001”;
***indicates “compared with the model group, p < 0.001”;
**indicates “compared with the model group, p < 0.01”; and
*indicates “compared with the model group, p < 0.05”.

The above examples are the preferred implementations of the disclosure, but the implementations of the disclosure are not limited by the above examples. Any other changes, modifications, substitutions, combinations, and simplifications made without departing from the spirit and principle of the disclosure should be equivalent substitute modes, and should be included in the scope of protection of the disclosure.

What is claimed is:

1. A Chinese herbal medicine composition, wherein the Chinese herbal medicine composition is any one of (b1) to (b4):
b1) comprising the following ingredients in parts by weight: 50 parts to 100 parts of avocado extract and 20 parts to 100 parts of ecklonia cava extract;
b2) comprising the following ingredients in parts by weight: 50 parts to 100 parts of avocado extract and 20 parts to 100 parts of perilla extract;
b3) comprising the following ingredients in parts by weight: 20 parts to 100 parts of ecklonia cava extract and 20 parts to 100 parts of perilla extract; and
b4) comprising the following ingredients in parts by weight: 50 parts to 100 parts of avocado extract, 20 parts to 100 parts of perilla extract and 20 parts to 100 parts of ecklonia cava extract.

2. The Chinese herbal medicine composition according to claim 1, wherein:
the Chinese herbal medicine composition is any one of (c1) to (c4):
c1) comprising the following ingredients in parts by weight: 60 parts to 90 parts of avocado extract and 30 parts to 90 parts of ecklonia cava extract;
c2) comprising the following ingredients in parts by weight: 60 parts to 90 parts of avocado extract and 30 parts to 90 parts of perilla extract;
c3) comprising the following ingredients in parts by weight: 30 parts to 90 parts of ecklonia cava extract and 30 parts to 90 parts of perilla extract; and
c4) comprising the following ingredients in parts by weight: 60 parts to 90 parts of avocado extract, 30 parts to 90 parts of perilla extract and 30 parts to 90 parts of ecklonia cava extract.

3. The Chinese herbal medicine composition according to claim 2, wherein:
the Chinese herbal medicine composition is any one of (d1) to (d4):
d1) comprising the following ingredients in parts by weight: 70 parts to 90 parts of avocado extract and 60 parts to 90 parts of ecklonia cava extract;
d2) comprising the following ingredients in parts by weight: 70 parts to 90 parts of avocado extract and 70 parts to 90 parts of perilla extract;
d3) comprising the following ingredients in parts by weight: 60 parts to 90 parts of ecklonia cava extract and 70 parts to 90 parts of perilla extract; and
d4) comprising the following ingredients in parts by weight: 70 parts to 90 parts of avocado extract, 70 parts to 90 parts of perilla extract and 60 parts to 90 parts of ecklonia cava extract.

4. The Chinese herbal medicine composition according to claim 1, wherein:
an unsaturated fatty acid content of the avocado extract ranges from 60 wt % to 80 wt %.

5. The Chinese herbal medicine composition according to claim 1, wherein:
a polyphenol content of the ecklonia cava extract ranges from 5 wt % to 25 wt %.

6. A product, comprising the Chinese herbal medicine composition according to claim 1.

7. The product according to claim 6, wherein:
the product comprises at least one of food and medicine.

8. The product according to claim 7, wherein:
the food comprises at least one of beverage, biscuit, candy and cake.

9. The product according to claim 7, wherein:
a dosage form of the medicine comprises at least one of capsule, tablet, granule, mixture, decoction, pill, electuary, dripping pill, medicinal tea, tincture and medicinal liquor.

10. The product according to claim 6, wherein:
the product has at least one function of (e1) to (e4):
e1) preventing and/or treating hearing impairment;
e2) reducing an ABR value;
e3) inhibiting generation of an inflammatory factor; and
e4) reducing a MDA content.
